(19) United States
(12) Patent Application Publication
(10) Pub. No.: US 2022/0395831 A1
(43) Pub. Date: Dec. 15, 2022

(54) DIRECT AND SCALABLE ISOLATION OF CIRCULATING EXTRACELLULAR VESICLES FROM WHOLE BLOOD USING CENTRIFUGAL FORCES

(71) Applicant: NANYANG TECHNOLOGICAL UNIVERSITY, Singapore (SG)

(72) Inventors: Han Wei HOU, Singapore (SG); Hui Min TAY, Singapore (SG); Sheng Yuan LEONG, Singapore (SG)

(21) Appl. No.: 17/770,976

(22) PCT Filed: Oct. 21, 2020
(86) PCT No.: PCT/SG2020/050601

§ 371 Date: Apr. 21, 2022

(30) Foreign Application Priority Data
Oct. 21, 2019 (SG) 10201909776U

(51) Int. Cl. B01L 3/00; G01N 33/49

(52) U.S. Cl. CPC B01L 3/502753; G01N 33/491; B01L 2300/0883; B01L 2300/0861; B01L 2400/0409

(57) ABSTRACT

A method herein to isolate exosomes includes providing a microfluidic device having a spiral-shaped channel in fluid communication with two inlet ports and at least two outlet ports. One of the two inlet ports is proximal to an inner wall of the spiral-shaped channel and the other is proximal to an outer wall thereof. At least one of the outlet ports is in fluid communication with a container for storing isolated exosomes. A blood sample and sheath fluid are introduced into the inlet ports proximal to the outer and inner walls, respectively, to form a diluted sample in the spiral-shaped channel and driven through for exosomes recovery in the container. The spiral-shaped channel in fluid communication with a first outlet port includes a first outlet channel connecting the spiral-shaped channel to the first outlet port and is longer than other outlet channels respectively connecting the spiral-shaped channel to the other outlet ports. A method of identifying diabetes mellitus is also disclosed herein.

### CROSS-REFERENCE TO RELATED APPLICATION

This application claims the benefit of priority of Singapore Patent Application No. 10201909776U, filed 21 Oct. 2019, the content of it being hereby incorporated by reference in its entirety for all purposes.

### TECHNICAL FIELD

The present disclosure relates to a method of isolating exosomes from blood.

The present disclosure also relates to a microfluidic device operable to carry out the method, and a method of identifying diabetes mellitus based on the microfluidic device and method.

### BACKGROUND

Extracellular vesicles (EVs), including exosomes (˜50-200 nm) and microvesicles (˜100 nm-1 μm), are produced by cells upon physiological or pathological cues and serve as mediators for intercellular communications. While circulating EVs in blood are promising diagnostics biomarkers in cancer and diabetes, isolation of blood-borne exosomes involves laborious ultracentrifugation or commercial precipitation kits with high protein contamination.

The complexity of blood with the high cellular components (˜50% v/v), and the similar size range between EVs and platelets (˜2-3 μm) present a huge technical challenge for EVs isolation. A current standard for EVs isolation may involve multi-step differential and ultracentrifugation (typically ˜1,000×g for 10 minutes to remove cellular components; ˜2,000×g for 20 minutes to obtain platelet-free plasma; ˜20,000×g for 60 minutes to pellet microvesicles; ˜175,000×g for 70 minutes to pellet exosomes). Such a standard may be commonly used to purify EVs, but the process is laborious and the EVs yield and purity may be highly dependent on user operation and blood collection method.

In one example, immunomagnetic bead-based capture of exosomes appears more effective, but may lead to biased analysis depending on the binding targets.

Commercial products based on filtration and/or precipitation are also available for isolating EVs from blood sera. However, despite their user-friendliness, purities are lower than the conventional/standard methods and there tends to be a risk of losing EVs functionalities after elution.

Several other exosomes isolation and detection microfluidics platforms have been developed, with the most common being affinity capture using well-established exosomal surface markers (CD81 or CD63) on microchannel surfaces or microbeads. These technologies were demonstrated with plasma or serum samples, which require additional sample processing (centrifugation) steps to deplete the blood cells. Throughput and flow rate tend to be low in these devices (˜4-20 μL min⁻¹) as they need to facilitate exosomes binding within channels or mixing with capture beads. This happens to limit direct whole blood processing as the large RBCs generates a background interference that significantly hinders binding of EVs to antibody-functionalized surfaces.

Another strategy was developed via size-based exclusion to isolate exosomes by membrane crossflow filtration, or microporous ciliated micropillars using silicon nanowires. These label-free approaches tend to be non-selective in trapping EVs which result in higher yield and unbiased analysis. Throughput may be scaled up easily with larger filtration footprint, but device operations are largely limited by clogging issues and low EVs recovery.

In another example, a microfluidic technology termed “High-resolution Dean Flow Fractionation (HiDFF)” for sub-micron binary particle sorting was developed. HiDFF exploits a non-equilibrium differential Dean migration of particles across a channel to achieve continuous size-based separation of small microparticles and nanoparticles (˜50 nm-1 μm). This provides for fractionation of small particles with high separation resolution at high throughput (˜70-100 μL min⁻¹) and purified particles may be continuously collected off chip for downstream analysis. However, such technology may be specific and does not sufficiently provide the resolution needed for exosome isolation.

There is thus a need to provide for a solution that addresses one or more of the limitations mentioned above. The solution should at least provide for a method of isolating EVs such as exosomes, and/or a multiplexed EVs fractionation tool with sub-300 nm separation resolution. The solution should also provide a capability of direct isolation of circulating exosomes from whole blood.

### SUMMARY

In a first aspect, there is provided for a method of isolating exosomes from blood, the method includes:

providing a microfluidic device having a spiral-shaped channel in fluid communication with (i) two inlet ports and (ii) at least two outlet ports, wherein one of the two inlet ports is proximal to an inner wall of the spiral-shaped channel and the other inlet port is proximal to an outer wall of the spiral-shaped channel, wherein at least one of the outlet ports is in fluid communication with a container configured to store isolated exosomes;

introducing a blood sample into the inlet port proximal to the outer wall and introducing a sheath fluid into the inlet port proximal to the inner wall to form a diluted sample in the spiral-shaped channel;

driving the diluted sample through the spiral-shaped channel; and

recovering the exosomes in the container,

wherein the at least two outlet ports include a first outlet port which is in fluid communication with the container configured to store the isolated exosomes,

wherein the spiral-shaped channel in fluid communication with the first outlet port includes a first outlet channel which connects the spiral-shaped channel to the first outlet port and is longer than other outlet channels respectively connecting the spiral-shaped channel to the other outlet ports.

In another aspect, there is provided for a microfluidic device operable to isolate exosomes from blood, the microfluidic device includes:

a spiral-shaped channel in fluid communication with (i) two inlet ports and (ii) at least two outlet ports, wherein one of the two inlet ports is proximal to an inner wall of the spiral-shaped channel and the other inlet port is proximal to an outer wall of the spiral-shaped channel; and

a container in fluid communication with at least one of the outlet ports, wherein the container is configured to store isolated exosomes,

wherein the at least two outlet ports include a first outlet port which is in fluid communication with the container configured to store the isolated exosomes,

wherein the spiral-shaped channel in fluid communication with the first outlet port comprises a first outlet channel which connects the spiral-shaped channel to the first outlet port and is longer than other outlet channels respectively connecting the spiral-shaped channel to the other outlet ports.

In another aspect, there is provided a method of identifying diabetes mellitus, the method includes:

providing a blood sample and introducing the blood sample into the microfluidic device described in various embodiments of the aspect mentioned above;

operating the microfluidic device; and

isolating exosomes according to the method described in various embodiments of the first aspect mentioned above to identify diabetes mellitus.

### BRIEF DESCRIPTION OF THE DRAWINGS

The drawings are not necessarily to scale, emphasis instead generally being placed upon illustrating the principles of the present disclosure. In the following description, various embodiments of the present disclosure are described with reference to the following drawings, in which:

FIG. 1A relates to a device schematic depicting the design of the 2-inlet, 4-outlet spiral microchannel of the present disclosure.

FIG. 1B illustrates separation of circulating EVs from whole blood. In FIG. 1B, under the influence of Dean vortices, small particles (EVs and platelets aₚ/h<0.07) migrate laterally towards inner wall. Near the inner wall, the innermost transient positions of the particles are determined by size-dependent wall-induced inertial lift forces (F_{WL}), which can be exploited for small particle separation with superior resolution.

FIG. 2A shows a fluorescent image of 50 nm beads and CFD predicted streamlines at the outlet region with 0x resistance channel length in outlet 1. ANSYS FLUENT was used to simulate streamlines from sample inlet.

FIG. 2B shows a fluorescent image of 50 nm beads and CFD predicted streamlines at the outlet region with 2.5x resistance channel length in outlet 1. ANSYS FLUENT was used to simulate streamlines from sample inlet.

FIG. 2C shows a cross-sectional view of streamlines at the outlet region with 0x resistance channel length in outlet 1. ANSYS FLUENT was used to simulate streamlines from sample inlet.

FIG. 2D shows a cross-sectional view of streamlines at the outlet region with 2.5x resistance channel length in outlet 1. ANSYS FLUENT was used to simulate streamlines from sample inlet.

FIG. 3A shows the average fluorescent composite images of 50 nm, 1 μm, 2 μm and 3 μm beads separation into different outlets.

FIG. 3B shows the flow cytometry analysis of bead separation efficiency in each outlet. O1, O2, O3, O4 denote for the four outlets.

FIG. 3C shows the scanning electron microscope (SEM) image analysis of bead separation efficiency in each outlet. O1, O2, O3, O4 denote for the four outlets. Scale bar denotes 1 μm.

FIG. 4A shows high speed stacked images of RBCs and platelets separation into outlets 3 and 4. The inset (box shown in middle image and enlarged view of the box in rightmost image) depicts platelets at 40× magnification. All samples were loaded equally (15 μL) except for plasma (0.3 μL).

FIG. 4B shows the nanoparticle tracking analysis (NTA), analyzing the size of EVs derived from ultracentrifugation (UC), HiDFF outlet 1 (O1) and outlet 2 (O2). All samples were loaded equally (15 μL) except for plasma (0.3 μL).

FIG. 4C shows the yield comparison of EVs derived from ultracentrifugation (UC), HiDFF outlet 1 (O1) and outlet 2 (O2). All samples were loaded equally (15 μL) except for plasma (0.3 μL).

FIG. 4D shows the western blot detection of exosomal marker CD9, annexin and histone expressions in plasma, HiDFF outlets and ultracentrifugation samples (microvesicles (MV) and exosomes). All samples were loaded equally (15 μL) except for plasma (0.3 μL).

FIG. 5 shows improvement of a reported HiDFF device design from 2-outlet to the present 4-outlet with an addition 2.5x resistance channel length, and their respective NTA size analysis results for outlet 1 eluent.

FIG. 6A shows plots of the size distribution in nucleotides (nt) and fluorescence intensity (FU) of total RNA in the 4 outlets. Size marker (internal standard) indicate 25 nt for RNA. RNA denotes ribonucleic acid.

FIG. 6B shows an overlay plot of exosomal RNA from ultracentrifugation, HiDFF outlet 1 and outlet 2 comparing RNA concentration.

FIG. 7A shows stacked devices (2- and 3-layered) of the present disclosure to achieve higher throughput.

FIG. 7B shows high speed images of 2 X blood separation in the top (left) and bottom (right) layer of a 2-layer device. Inset shows similar separation of fluorescent 50 nm beads into outlet 1.

FIG. 7C depicts NTA results from outlet 1 of single (left plot) and 2-layered (right plot) devices showing similar EVs size cut-off.

FIG. 7D shows fluorescent imaging of 1 μm beads sorting in a 3-layered device, illustrating similar flow profiles, with majority exiting from outlet 2.

FIG. 8A compares the 1^{st} generation 4-outlet HiDFF device to the present 2^{nd} generation 2-outlet ExoDFF devices and a 4-spiral high throughput ExoDFF (ExoDFF^{HT}) device of the present disclosure. The abbreviation “Gen” and “ExoDFF” denote for the terms “generation” and “Exosomes Dean Flow Fractionation”, respectively. The 2^{nd} generation ExoDFF and the ExoDFF^{HT} have the longer first outlet channel as described in various embodiments herein.

FIG. 8B depicts fluorescent composite images showing the separation of 50 nm (leftmost fluorescent composite image-green), 500 nm (center fluorescent composite image-yellow), 1 μm (rightmost fluorescent composite image-red) fluorescent beads in outlet 1 (O1) and outlet 2 (O2) of the 2^{nd} Gen ExoDFF device. Scale bar denotes 200 μm. FIG. 8B also shows average intensity linescans of the fluorescent beads along the device channel width at optimal flow condition (Reynolds number is 42).

FIG. 9A shows a photograph of the 4-spiral ExoDFF^{HT} device filled with red dye for visualization. Quantification of 200 nm separation efficiency in outlet 1 (O1) of 1st Gen (4-outlet) HiDFF, 2nd Gen (2-outlet) ExoDFF and 2^{nd} Gen ExoDFF^{HT} devices indicating similar separation efficiency at two different flow rates.

FIG. 9B shows high speed images of ExoDFF^{HT} outlet regions indicating similar flow profiles of blood cells and their removal into the larger outer wall waste outlet (O2).

FIG. 10A presents the NTA analysis of particle concentration from all 4 spirals of ExoDFF^{HT} and UC isolated small EVs. All samples were loaded with equal volume (15 μL) except plasma (0.5 μL).

FIG. 10B demonstrates the higher EVs separation efficiency derived from 1st Gen HiDFF, 2nd Gen ExoDFF and ExoDFF^{HT} devices compared to UC. All samples were loaded with equal volume (15 μL) except plasma (0.5 μL).

FIG. 10C shows the Western blot detection of exosomal markers TSG101, flotillin-1 and CD9, as well as albumin and apoA-1 for blood plasma. All samples were loaded with equal volume (15 μL) except plasma (0.5 μL).

FIG. 11A shows a schematic of the top and cross-sectional view of 2-inlet, 2-outlet half-loop ExoDFF device with 300 μm width.

FIG. 11B shows fluorescent composite images on the separation of 50 nm (green), 500 nm (yellow), 1 μm (red) beads in PBS into O1 and O2 at different flow rates (1:10 sample to buffer ratio).

FIG. 11C shows fluorescent composite images on the separation of 50 nm (green), 500 nm (yellow), 1 μm (red) beads mixed with 1× diluted whole blood (with PBS) into O1 and O2 at different flow rates (1:10 sample to buffer ratio).

FIG. 11D demonstrates NTA of particle concentration and size of inlet 1× diluted blood sample, O1 and O2 eluent at two different flow rates.

FIG. 12A shows a schematic of the top and cross-sectional view of 2-inlet, 2-outlet half-loop ExoDFF device with 500 μm width.

FIG. 12B shows fluorescent composite images on the separation of 50 nm (green), 500 nm (yellow), 1 μm (red) beads in PBS into O1 and O2 at different flow rates (1:20 sample to buffer ratio).

FIG. 13A shows high speed images illustrating RBCs and platelets separation into ExoDFF outlet 3 (O3) and outlet 4 (outlet) as waste outlets. Inset (blue box—center image and magnified rightmost image) depicts platelet flow position at 40× magnification.

FIG. 13B shows particle concentration and size distribution plots of ExoDFF O1 and UC exosomes (UC exo), and ExoDFF O2 and UC MV using NTA.

FIG. 13C shows EV concentrations in different outlets of ExoDFF after separation (n=3).

FIG. 13D compares EV separation efficiencies between ExoDFF and UC (n=3).

FIG. 13E depicts the EV yield comparison (fold-change) between UC exo and ExoDFF O1 (n=7). **P<0.01 by unpaired Mann-Whitney test.

FIG. 13F shows the Western blot detection of exosomal markers TSG101, flotillin and CD9, and apoA-1 for blood plasma. All samples were loaded with equal volume (15 μL) except plasma (0.5 μL).

FIG. 13G is a transmission electron microscopy (TEM) image of the EV with cup-shaped morphology isolated from ExoDFF O1. Scale bar denotes 100 nm.

FIG. 13H is a MicroRNA quantitation of EV from ExoDFF O1, O2 and UC Exo. Plot shows the size distribution in nucleotides (nt) and fluorescence intensity (FU) of total RNA. Size marker (internal standard) indicates 25 nt for RNA.

FIG. 14A is a comparison of EV yield per mL of whole blood (WB), isolated using ExoDFF from healthy (n=7) and T2DM subjects (n=5). Results are also compared with those for UC in all cases, with data are presented using mean±s.e.m.*P<0.05 and **P<0.01.

FIG. 14B shows correlation of EV yield with HbA1c (%).

FIG. 15 shows a prototype developed based on the present methods and incorporating the present microfluidic device. The prototype is non-invasive for exosome sampling and allows for multiple sample collection over time and continuous monitoring of disease progression and response to therapy. The prototype can be automated in that it circumvents/minimizes human intervention throughout the separation process, thereby reducing human error as well. Moreover, the results are reproducible over multiple runs because of stable operation parameters rendered by the present device. The prototype can be low cost as it is operable based on a continuous-flow basis which reduces lead time compared to methods involving multiple processes, thereby cutting down operation cost and overall separation time. The prototype is reliable as it can separate exosomes without involving antibody labels or compromising the targets for sampling. Conversely, methods involving labelling tend to be costly, time-consuming and may be easily biased.

FIG. 16 compares the present method and microfluidic device (see center and rightmost column of images), which involves one longer outlet channel connecting the spiral-shaped channel to the first outlet port, compared to a method and microfluidic device where the outlet channels to all outlets ports are of the same length. The method and microfluidic device, represented by the rightmost image, provided the most desirable exosomes isolation results.

### DETAILED DESCRIPTION

The following detailed description refers to the accompanying drawings that show, by way of illustration, specific details and embodiments in which the present disclosure may be practiced.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The present disclosure introduces a strategy for rapid and continuous isolation of extracellular vesicles, such as exosomes, from whole blood directly, which facilitates and improves the influence of centrifugal-induced Dean migration in, for example, a spiral microfluidic device. The strategy allows scalable, single-step size-based purification of exosomes without the need for ultracentrifugation or additional labelling or processing steps.

The strategy includes a method and a device which provide improved separation or fractionation resolution to isolate extracellular vesicles, such as exosomes, in a sample. The sample may be a blood sample. The present method and microfluidic device provide several advantages over existing methods as it is label-free, able to process whole blood directly at high throughput, and workable with micro-sized and nano-sized features without clogging. The terms “separation” and “fractionation” herein may be used interchangeably.

Details of various embodiments of the present method and microfluidic device, and advantages associated with the various embodiments are now described below. The various embodiments and advantages are also demonstrated through the examples provided further below herein.

In the present disclosure, there is provided a method of isolating exosomes from blood. The method includes providing a microfluidic device having a spiral-shaped channel in fluid communication with (i) two inlet ports and (ii) at least two outlet ports, wherein one of the two inlet ports is proximal to an inner wall of the spiral-shaped channel and the other inlet port is proximal to an outer wall of the spiral-shaped channel. At least one of the outlet ports may be in fluid communication with a container configured to store isolated exosomes.

The present method may include introducing a blood sample into the inlet port proximal to the outer wall and introducing a sheath fluid into the inlet port proximal to the inner wall to form a diluted sample in the spiral-shaped channel. The blood sample and sheath fluid can be introduced by means of a syringe, by dipping the device into a sample, or other means.

With one of the two inlet ports proximal to an inner wall of the spiral-shaped channel and the other inlet port proximal to an outer wall of the spiral-shaped channel, this facilitates and improves the influences of inertial focusing separation in a curved channel (i.e. the spiral-shaped channel). Such inertial focusing separation in a curved channel may be termed herein “Dean flow separation”. Said differently, the described arrangement of the two inlets port helps to set up a flow condition in a spiral-shaped channel such that the forces acting on the exosomes advantageously renders the exosomes in a sample to be completely isolated.

Moreover, with one of the two inlet ports proximal to an inner wall of the spiral-shaped channel and the other inlet port proximal to an outer wall of the spiral-shaped channel, the introduction of the blood sample and sheath fluid in the manner as described herein also assists for the exosomes to be significantly influenced by Dean flow, such that the exosomes get channeled to the inner wall of the spiral-shaped channel before exiting the spiral-shaped channel. In other words, the exosomes experience a centripetal force that drives the exosomes toward a point as the “center” of a curvature of the spiral-shaped channel as the exosomes flow in the spiral-shaped channel. Following this flow configuration, it can be easily understood that the inner wall of the spiral-shaped channel is the wall proximal to the “center” and the outer wall of the spiral-shaped channel is the wall that is away from the “center”. The term “proximal” herein includes within its meaning “near”, “at”, or “in vicinity of”.

The term “sheath fluid” herein refers to a variety of fluids, including aqueous or nonaqueous fluids and/or fluids that may include additional material components, e.g., soluble chemical components or suspensions or emulsions of at least partially insoluble components. As a non-limiting example, the sheath fluid can be a buffer that is compatible with blood cells, such as phosphate-buffered saline (abbreviated PBS). The term “buffer” herein means any compound or combination of compounds that control the pH of the environment in which they are dissolved or dispersed. Concerning the pH value, buffers diminish the effect of acids or basis added to the buffer solution. Buffers generally can be broken into two categories based upon their solubility. Both categories of buffer separately, or in combination, can be employed. “Water-soluble buffers” typically have a solubility in water of at least 1 gm in 100 ml, at least 1 gm in 75 ml, or at least 1 gm in 30 ml, etc. Examples of water-soluble buffers include, but are not limited to PBS, meglumine, sodium bicarbonate, sodium carbonate, sodium citrate, calcium gluconate, disodium hydrogen phosphate, dipotassium hydrogen phosphate, tripotassium phosphate, sodium tartarate, sodium acetate, calcium glycerophosphate, tromethamine, magnesium oxide or any combination of the foregoing. “Water-insoluble buffers” typically have a solubility in water less than 1 gm in 1,000 ml, less than 1 gm in 5,000 ml, or less than 1 gm in 10,000 ml, etc. Examples of water-insoluble buffers include, but are not limited to magnesium hydroxide, aluminum hydroxide, dihydroxy aluminum sodium carbonate, calcium carbonate, aluminum phosphate, aluminum carbonate, dihydroxy aluminum amino acetate, magnesium oxide, magnesium trisilicate, magnesium carbonate, and combinations of the foregoing. Buffer can also be supplemented with supporting agents, such as salts, detergents, BSA (bovine serum albumin), etc.

The present method may include driving the diluted sample through the spiral-shaped channel, and recovering the exosomes in the container, wherein the at least two outlet ports comprise a first outlet port which is in fluid communication with the container configured to store the isolated exosomes. The diluted sample can be driven by a force of capillary attraction. Alternatively, the diluted sample can be driven any pump, by electrical forces, or by other means for driving samples through the inlets, spiral-shaped channel, and out of the outlets.

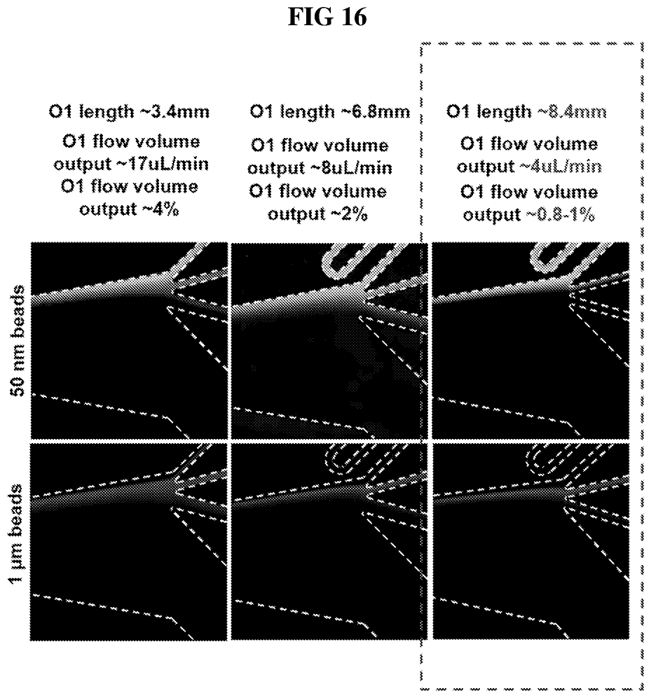

In the present method, the spiral-shaped channel in fluid communication with the first outlet port includes a first outlet channel which connects the spiral-shaped channel to the first outlet port and is longer than other outlet channels respectively connecting the spiral-shaped channel to the other outlet ports. An “outlet channel” herein refers to a channel that connects the spiral-shaped channel to an outlet port. For example, the first outlet channel is the channel that connects the spiral-shaped channel to the first outlet port from which the exosomes exit. The first outlet channel is termed herein as such, as it is the channel which connects to and is in fluid communication with the first outlet port. Accordingly, a second/third/fourth outlet channel refers to respective channel that connects to and is in fluid communication with the second/third/fourth outlet port, respectively. The first outlet channel is demonstrated in the form of a serpertine channel only by way of a non-limiting example (see examples section herein, also see, e.g. FIG. 16). Other designs that render the first outlet channel longer than the other outlet channels may be used. The first outlet channel can be shaped as a serpentine channel to render a longer flow path for the exosomes to exit. Advantageously, the longer first outlet channel renders more fluidic resistance that imparts better separation resolution for exosomes to be completely isolated in the first outlet. This is due to a smaller flow volume output into the first outlet channel which advantageously elute specifically exosomes, wherein the exosomes are positioned at and/or close to the inner wall while preventing any larger particles from exiting through first outlet channel and first outlet port. In various embodiments, as the first outlet channel may be the only channel having a length longer than the other outlet channels, the first outlet channel may be specifically referred to as a “first connecting channel”, wherein the term “first” in this expression signifies the connection of the spiral-shaped channel to the first outlet port.

In various embodiments, the first outlet channel may have a length ranging from 0.5 cm to 1.5 cm, 1 cm to 1.5 cm, etc. This can confer an advantage of collecting a small fraction (e.g. —0.5-4%) of the entire volume output and collecting the fluid exiting from the inner wall region.

Further advantageously, the present method avoids the need for a centrifugation step (i.e. use of a centrifugation machine). Said differently, the method does not include a centrifugation step.

In various embodiments, introducing the blood sample and the sheath fluid may include introducing the sheath fluid at a higher flow rate compared to a flow rate for introducing the blood sample. For example, introducing the blood sample and the sheath fluid may include introducing the blood sample into the inlet port proximal to the outer wall and introducing the sheath fluid into the inlet port proximal to the inner wall at a flow rate ratio of 1:5 to 1:50, 1:10 to 1:50, 1:20 to 1:50, 1:30 to 1:50, 1:40 to 1:50, etc. These flow rate ratios help to initially confine the blood sample at the outer channel wall so as to have a controlled and tighter Dean-induced lateral migration of exosomes towards the inner wall for efficient exosomes separation.

In various embodiments, the spiral-shaped channel may be defined as having a width ranging from 150 μm to 500 μm, 200 μm to 500 μm, 250 μm to 500 μm, 300 μm to 500 μm, 350 μm to 500 μm, 400 μm to 500 μm, 450 μm to 500 μm, etc. These channel width dimensions are suitable to process high cell concentration samples (e.g. whole blood) with minimal channel clogging issues.

In various embodiments, the spiral-shaped channel may be defined as having a height ranging from 30 μm to 100 μm, 40 μm to 100 μm, 50 μm to 100 μm, 60 μm to 100 μm, 70 μm to 100 μm, 80 μm to 100 μm, 90 μm to 100 μm, etc. These channel height dimensions help to prevent the larger cells (˜5 to 20 um) from flowing close to the inner wall where the exosomes migrate to or are located.

In various embodiments, the spiral-shaped channel may be defined as having a length ranging from 3 cm to 10 cm, 4 cm to 10 cm, 5 cm to 10 cm, 6 cm to 10 cm, 7 cm to 10 cm, 8 cm to 10 cm, 9 cm to 10 cm, etc. These channel length dimensions render formation of the secondary Dean vortices in the spiral-shaped channel that are sufficiently stable to induce exosome migration to inner wall of the spiral-shaped channel.

In various embodiments, the spiral-shaped channel may be defined as having a width to height aspect ratio ranging from 3 to 7, 4 to 7, 5 to 7, 6 to 7, etc. These aspect ratios help to prevent the larger cells (e.g. —5 to 20 urn) from flowing close to the inner wall where the exosomes migrate to or are located.

In various embodiments, the spiral-shaped channel may be defined as having a radius curvature ranging from 0.3 cm to 1 cm, 0.4 cm to 1 cm, 0.5 cm to 1 cm, 0.6 cm to 1 cm, 0.7 cm to 1 cm, 0.8 cm to 1 cm, 0.9 cm to 1 cm, etc. These radii of curvature render formation of secondary Dean vortices in the spiral-shaped channels. The radius of curvature of the spiral-shaped channel is the distance measured from a center of the spiral-shaped channel's cross-section to the “centripetal” center, such that the radius measured is orthogonal to the motion of fluid flowing in the spiral-shaped channel. In other words, the centripetal center is a fixed point of the center of curvature of the path.

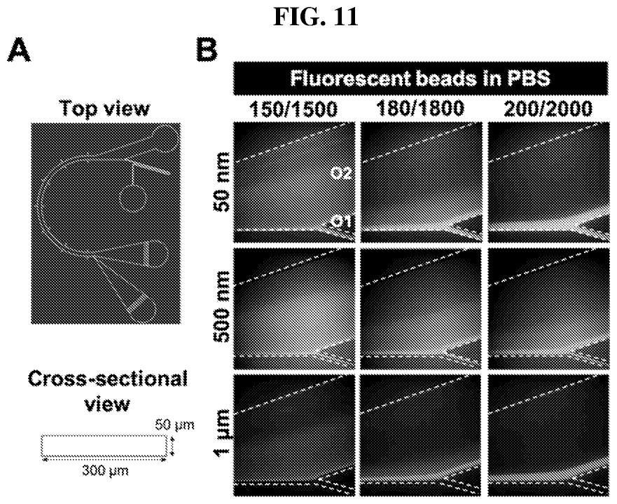

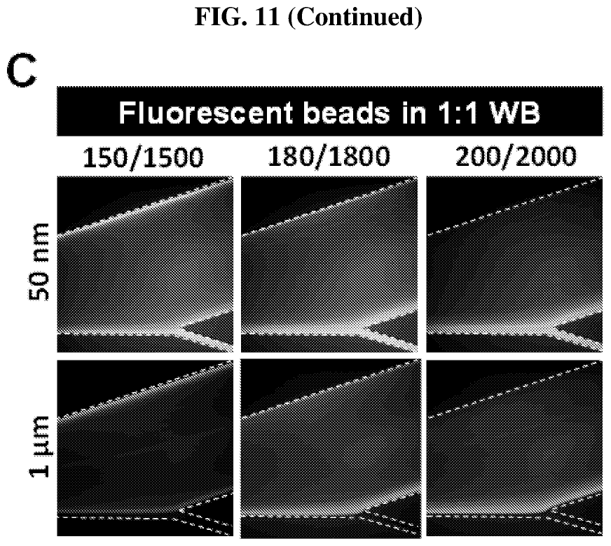

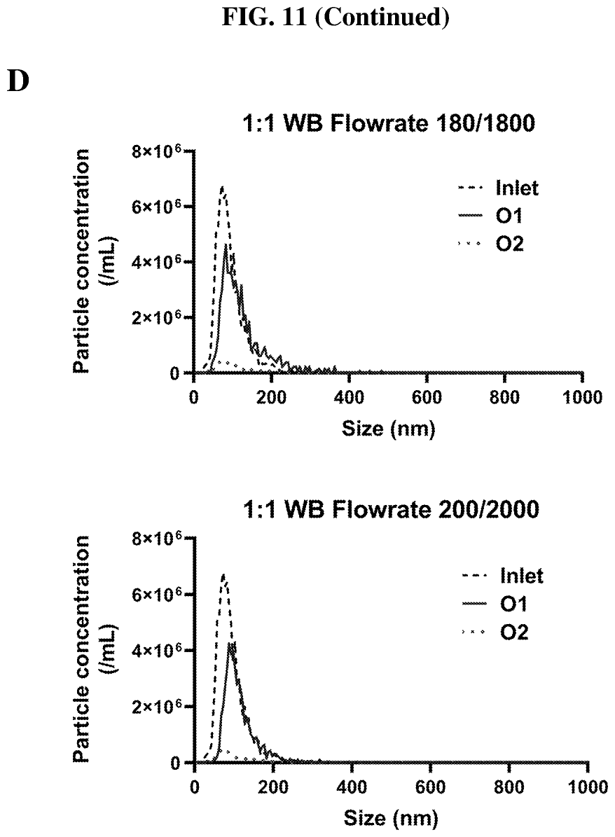

The spiral-shaped channel may be termed herein a “spiral-shaped microchannel” due to one or more dimensions being in the micron-sized range. The spiral-shaped channel may have one or more of the dimensions and/or ratios mentioned above. In certain embodiments, the spiral-shaped channel may be a semi-spiral-shaped channel. This means the spiral-shaped channel is a channel forming a semi-circle (see e.g. FIG. 11A and/or 12A). Such semi-spiral-shaped channel may be exchangeably referred to herein as a “half-loop”. The semi-spiral-shaped channel may have a length ranging from 5 mm to 25 mm, 10 mm to 25 mm, 15 mm to 25 mm, 20 mm to 25 mm, 5 mm to 10 mm, 5 mm to 15 mm, 5 mm to 20 mm, 10 mm to 20 mm, 10 mm to 15 mm, 15 mm to 20 mm, etc. Advantageously, the semi-spiral-shaped channel increases flow rate of a fluid through the spiral-shaped channel.

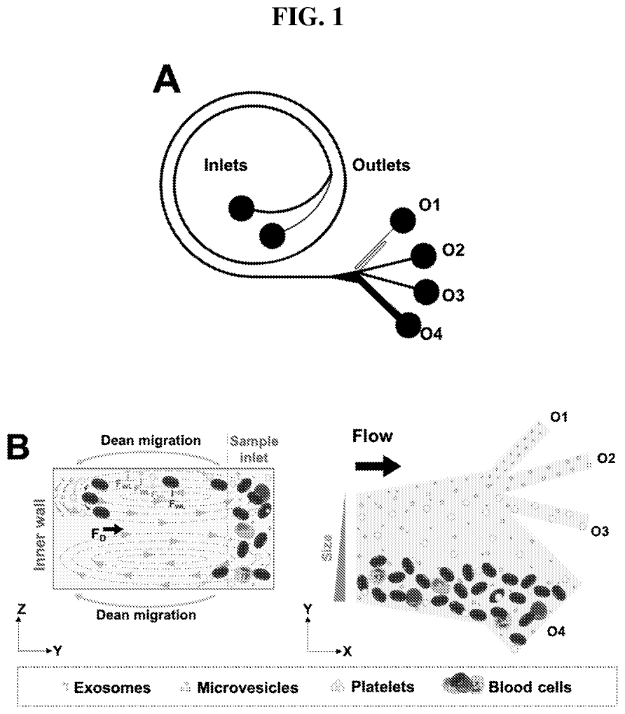

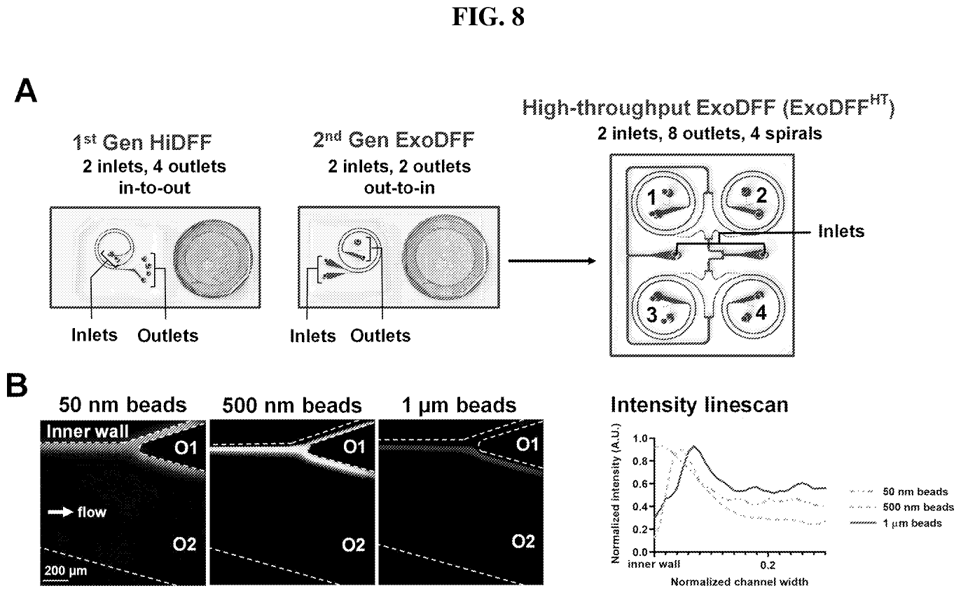

In various embodiments, the two inlet ports may be arranged in a manner where the spiral-shaped channel horizontally spirals around the inlet ports and the at least two outlet ports may be arranged away from the spiral-shaped channel (see e.g. FIG. 1A, FIG. 8A). In certain embodiments, the two inlet ports may be arranged away from the spiral-shaped channel and the at least two outlet ports may be arranged in a manner where the spiral-shaped channel horizontally spirals around the at least two outlet ports (see e.g. FIG. 8A).

In various embodiments, driving the diluted sample may include driving the diluted sample to flow in the spiral-shaped channel to have a Reynolds number ranging from 20 to 100, 30 to 100, 40 to 100, 50 to 100, 60 to 100, 70 to 100, 80 to 100, 90 to 100, etc., and/or a Dean number ranging from 2 to 10, 3 to 10, 4 to 10, 5 to 10, 6 to 10, 7 to 10, 8 to 10, 9 to 10, etc. As used herein, Reynolds number means ρυL/μ, wherein p represents density of a liquid, υ represents velocity of the liquid, represents characteristic length of a flow channel, and μ represents viscosity of the liquid. As used herein, Dean number refers to a product of the Reynolds number (based on axial flow υ through a channel of diameter D) and the square root of the curvature ratio, i.e. Re√[D/(2R_{c})], wherein R_{c} the radius of curvature of the path of the channel.

In certain embodiments, the at least two outlet ports may include four outlet ports. The number of outlet ports may depend on the components, other than exosomes, to be isolated from the sample.

In various embodiments, the spiral-shaped channel may gradually expand or furcate to a width of 500 μm to 3000 μm. In other words, the end of the spiral-shaped channel that is connected to the one or more outlet ports may furcate into the outlet channels that connect to their respective outlet ports. In certain embodiments, the first outlet port and/or first outlet channel may have a width ranging from 20 μm to 100 μm and the other outlet ports and/or other outlet channels then have a width that adds up to 500 μm to 3000 μm. As one example, the spiral-shaped channel may gradually expand or furcate to a width of 1000 μm, wherein the width of the first port outlet port and/or first outlet channel may be 50 μm, the width of the second outlet port and/or second outlet channel may be 50 μm, the width of the third outlet port and/or third outlet channel may be 50 μm, the width of the fourth outlet port and/or fourth outlet channel may be 4^{th} outlet may be 800 μm.

In various embodiments, the first outlet port may have a width ranging from 20 μm to 100 μm, 30 μm to 100 μm, 40 μm to 100 μm, 50 μm to 100 μm, 60 μm to 100 μm, 70 μm to 100 μm, 80 μm to 100 μm, 90 μm to 100 μm, etc. Such widths assist in size-based separation of exosomes, increasing the yield of exosomes isolated.

The present disclosure also provides for a microfluidic device operable to isolate exosomes from blood. Embodiments and advantages described in various embodiments for the method of the first aspect can be analogously valid for the present microfluidic device subsequently described herein, and vice versa. As the various embodiments and advantages have already been described above and examples demonstrated herein, they shall not be iterated for brevity.

The microfluidic device includes a spiral-shaped channel in fluid communication with (i) two inlet ports and (ii) at least two outlet ports, wherein one of the two inlet ports is proximal to an inner wall of the spiral-shaped channel and the other inlet port is proximal to an outer wall of the spiral-shaped channel, and a container in fluid communication with at least one of the outlet ports, wherein the container is configured to store isolated exosomes.

In various embodiments, the at least two outlet ports may include a first outlet port which is in fluid communication with the container configured to store the isolated exosomes, wherein the spiral-shaped channel in fluid communication with the first outlet port includes a first outlet channel which connects the spiral-shaped channel to the first outlet port and is longer than other outlet channels respectively connecting the spiral-shaped channel to the other outlet ports.

In various embodiments, the first outlet channel may have a length ranging from 0.5 cm to 1.5 cm. Other lengths of the first outlet channel are already described above in embodiments relating to the method of the first aspect.

In various embodiments, the inlet port proximal to the inner wall of the spiral-shaped channel is operable to introduce the sheath fluid at a higher flow rate than the inlet port proximal to the outer wall of the spiral-shaped channel.

In various embodiments, the inlet port proximal to the outlet wall of the spiral-shaped channel and the inlet port proximal to the inner wall of the spiral-shaped channel are operable to introduce the blood sample and the sheath fluid at a flow rate ratio of 1:5 to 1:50. Other flow rate ratios are already described above in embodiments relating to the method of the first aspect.

The spiral-shaped channel may be defined as having a width ranging from 150 μm to 500 μm, a height ranging from 30 μm to 100 μm, a length ranging from 3 cm to 10 cm, a width to height aspect ratio ranging from 3 to 7, and/or a radius curvature ranging from 0.3 cm to 1 cm. Other dimensions and ratios are already described above in embodiments relating to the method of the first aspect.

The spiral-shaped channel may be a semi-spiral-shaped channel. The semi-spiral-shaped channel may have a length as described in various embodiments of the method of the first aspect.

In various embodiments, the two inlet ports may be arranged in a manner where the spiral-shaped channel horizontally spirals around the inlet ports and the at least two outlet ports may be arranged away from the spiral-shaped channel. In certain embodiments, the two inlet ports may be arranged away from the spiral-shaped channel and the at least two outlet ports may be arranged in a manner where the spiral-shaped channel horizontally spirals around the at least two outlet ports.

The at least two outlet ports may include four outlet ports.

The spiral-shaped channel may gradually expand or furcate to a width of 500 μm to 3000 μm.

The first outlet port may have a width ranging from 20 μm to 100 μm.

The present microfluidic device may be used in ex vivo and/or in vitro identification of diabetes, such as diabetes mellitus. As described above and in the examples section herein, a sample such as blood sample may be drawn from a subject. Then, without the need for the subject to be present, the blood sample may be, injected as an example, into the present microfluidic device.

The present disclosure also provides for a method of identifying diabetes, such as diabetes mellitus, the method may include providing a blood sample and introducing the blood sample into the microfluidic device described according to various embodiments above, operating the microfluidic device, and isolating exosomes according to the method described in various embodiments of the first aspect to identify diabetes mellitus.

Embodiments and advantages described for the present method of the first aspect and for the microfluidic device can be analogously valid for the present method of identifying diabetes mellitus described herein, and vice versa. As the various embodiments and advantages have already been described above and examples demonstrated herein, they shall not be iterated for brevity. Demonstration/Application of the present method and microfluidic device to identify type 2 diabetes are described in the examples section below.

The word “substantially” does not exclude “completely” e.g. a composition which is “substantially free” from Y may be completely free from Y. Where necessary, the word “substantially” may be omitted from the definition of the present disclosure.

In the context of various embodiments, the articles “a”, “an” and “the” as used with regard to a feature or element include a reference to one or more of the features or elements.

In the context of various embodiments, the punctuation “—”, the term “about” or “approximately” as applied to a numeric value encompasses the exact value and a reasonable variance.

As used herein, the term “and/or” includes any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

### Examples

The present disclosure relates to a method and a microfluidic device operable to isolate extracellular vesicles, particularly or specifically exosomes, from blood.

The present method and microfluidic device may involve, for example, the use of a 2-inlet and at least a 2-outlet (e.g. 4-outlet) system spiral microchannel that can demonstrate multiplexed size-based fractionation of EVs (exosomes (˜50-200 nm) and microvesicles (˜100 nm-1 μm), platelets (˜2 μm) and blood cells (>8 μm) into the different outlets. The present method and microfluidic device provide for a label-free, low-cost EVs purification approach with desirably high throughput (˜20 μL undiluted whole blood (WB)/min), are scalable by device stacking, and offer higher exosomes yield as compared to ultracentrifugation. WB denotes whole blood. The spiral-shaped channel is termed herein a “spiral-shaped microchannel” due to its micron-sized dimension. The terms “spiral” and “spiral-shaped” herein are used exchangeably to describe the structure of the channel.

The present method and microfluidic device involve a spiral-shaped or curvilinear channel in which a sample suspected to contain exosomes can flow. The spiral-shaped or curvilinear channel may have at least a first end and a second end, wherein the spiral-shaped channel can have at least two inlet ports proximal to (i.e. at or near) the first end and at least two outlet ports proximal to the second end, wherein the at least two outlet ports contain a first outlet port (O1) that can be in fluid communication with an additional channel length, which provides for more fluidic resistance, to render sub-300 nm separation resolution for the separation of exosomes. This additional channel length may be termed herein a “first connecting channel” or a “first outlet channel” as described above.

In certain embodiments, the additional channel length connected to a first outlet port (O1) can be, for example, 0.5 cm to 1.5 cm. The additional channel length connected to the first outlet port (O1) can be, for example, 0.5 cm to 1.5 cm, and have a flow rate of 1 to 15 uL/min for example. The first outlet port (O1) is proximal to or located at the inner wall region. In other words, the first outlet port is positioned to receive exosomes that flows proximal to the inner wall region of the spiral-shaped microchannel The outlet port (O1) will collect a fraction (˜0.5 to 4%) of the total volume output.

In various embodiments, the microfluidic device may have more than one flow channel to allow multiplexing.

Advantageously, the present microfluidic device allows for sub-300 nm separation resolution. Advantageously, the proposed microfluidic device is able to completely isolate exosomes from whole blood.

The present method and microfluidic device may be referred to herein as “HiDFF” technology and/or “ExoDFF” technology. The present method and microfluidic device are described in further details, by way of non-limiting examples, as set forth below.

### Example 1: Present Method and Microfluidic Device Design

The 2-inlet, 4-outlet spiral microdevice (300 μm (w)×50 μm (h)) was fabricated in polydimethylsiloxane (PDMS) using standard soft lithography techniques and has a radius of 0.5-0.6 cm and a total length of ˜6.5 cm (FIG. 1A). The sample (50 μm wide) and sheath (250 μm wide) inlets are fixed at the outer and inner wall of the channel, respectively. At the outlet bifurcation, the channel gradually expands into a 4-outlet-system with optimized non-uniform fluidic resistance to facilitate EVs separation based on subtle size differences. The outlet closest to the channel inner wall is designed to collect exosomes (outlet 1) and larger microvesicles (outlet 2), while larger platelets and blood cells are collected at the outlet nearer to the channel outer wall (outlets 3 and 4, respectively).

During device operation, diluted WB (1:1) is perfused through the outer inlet while sheath buffer (1× phosphate buffer saline (PBS)) is perfused through the inner inlet at a higher flow rate (1:10) to confine the sample stream near the outer wall. As smaller blood components (platelets and EVs, particle size (aₚ)/hydraulic diameter (Dₕ) <0.07) traverse through the channel, they experience lateral drag forces (F_{D}) and migrate towards the inner wall due to influence of Dean vortices (FIG. 1B). Near the inner wall, they occupy differential innermost transient (non-equilibrium) positions due to size-dependent wall-induced inertial lift force (F_{WL}) for high-resolution size fractionation into outlets 1-3. Larger blood cells, experiencing significant Stoke's drag, remain closer to the outer wall and exit via outlet 4 (see right image in FIG. 1B).

### Example 2: Device Operation

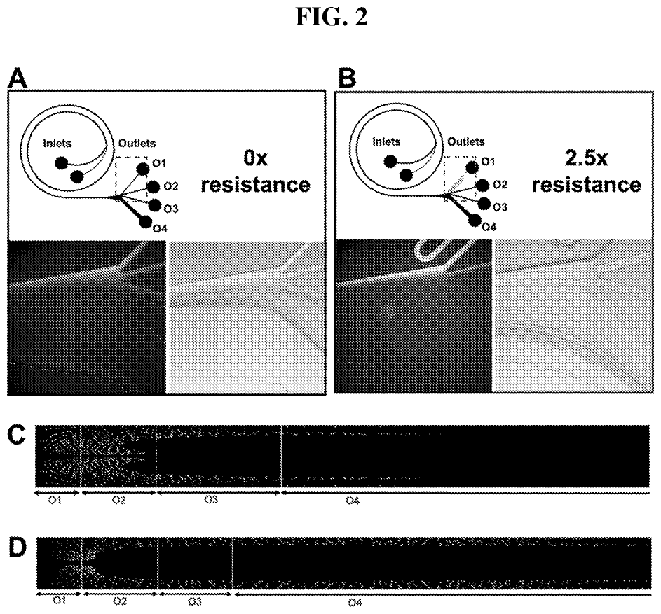

Computational fluid dynamics (CFD) modelling using ANSYS FLUENT was conducted to study the outlet design. Exosomes were assumed to behave like fluid particles due to its tiny size (<200 nm) and their streamlines were tracked starting from sample inlet and throughout the channel. The channel resistance of outlet 1 was first investigated by varying its channel length (denoted as 0x and 2.5x resistance). The flow trajectories of 50 nm fluorescent beads obtained experimentally, and streamlines profile predicted using CFD were then compared between different channel length of outlet 1 (FIGS. 2A and 2B). As the hydraulic resistance is higher with the longer channel length in 2.5x resistance design, flow rate into outlet 1 is reduced by ˜2.11 fold which enables collection of a subset of fluid streamlines (denoted orange line, i.e. the streamline(s) towards the top of the bottom right image in FIG. 2B) closest to the inner wall. Despite the slightly larger spread of streamlines across 4 outlets in the 2.5x resistance design. It can be experimentally seen that the 50 nm beads band (mimicking exosomes) were efficiently collected into outlet 1. This was further confirmed by cross-sectional view of CFD streamlines profile (FIGS. 2C and 2D), which showed tighter focusing of streamlines close to outlet 1 in the 2.5x resistance design. On the contrary, fluid streamlines occupy a broader position in the 0x resistance design, as the majority of streamlines enter both outlets 1 and 2, leading to significant loss of exosomes into outlet 2.

### Example 3: Results and Discussion of Present Device

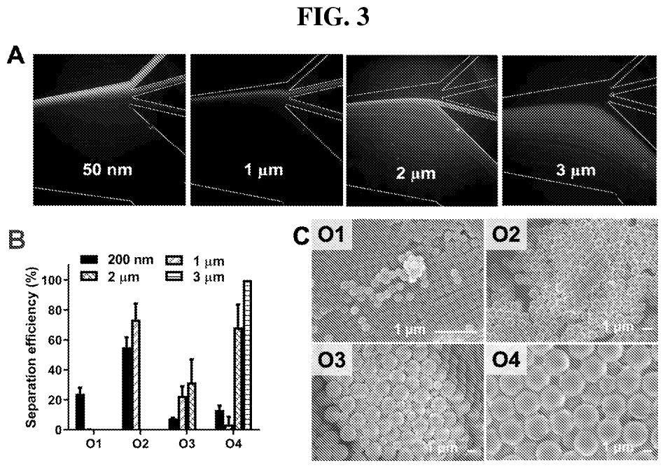

To characterize the present device and to study the flow rate conditions, fluorescent polystyrene microbeads (aₚ/h<0.07) of defined sizes (e.g. 50 nm, 1 μm, 2 μm, 3 μm) were used to visualize streamline positions and successful multiplexed bead separation (FIG. 3A) was demonstrated. Bead separation efficiency was also quantified by flow cytometry and scanning electron microscopy (FIGS. 3B and 3C) to confirm size-based bead fractionation into different outlets.

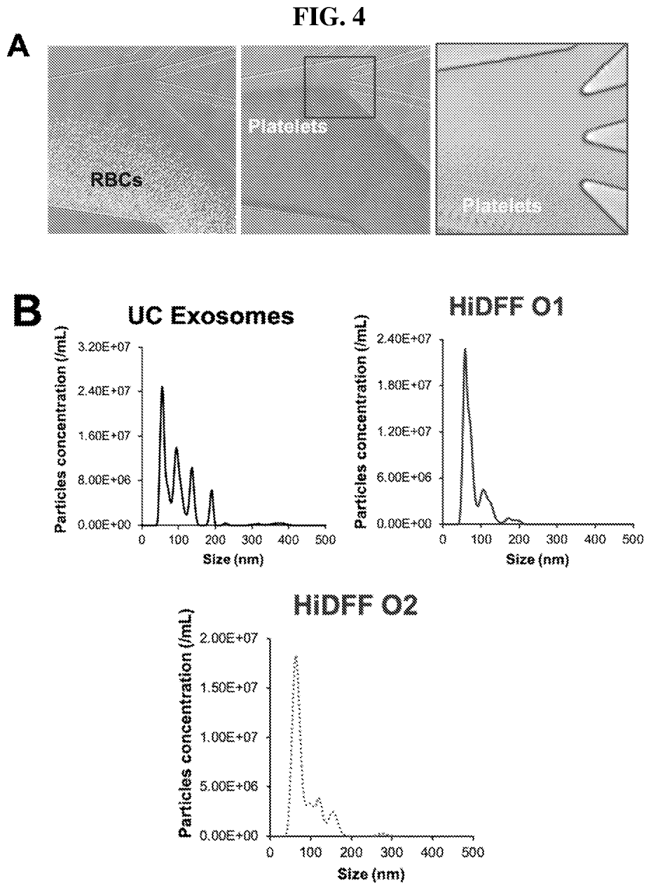

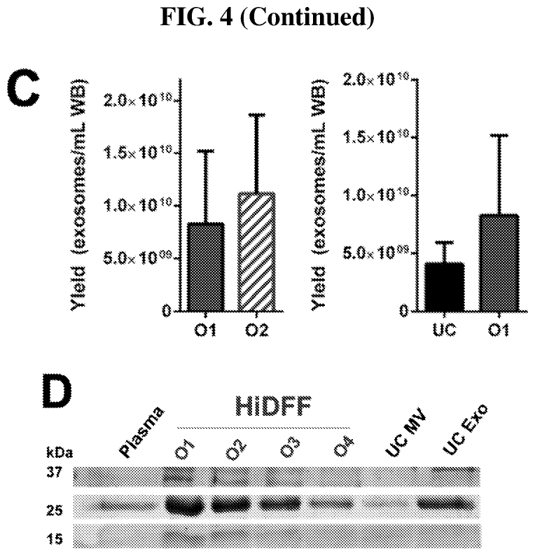

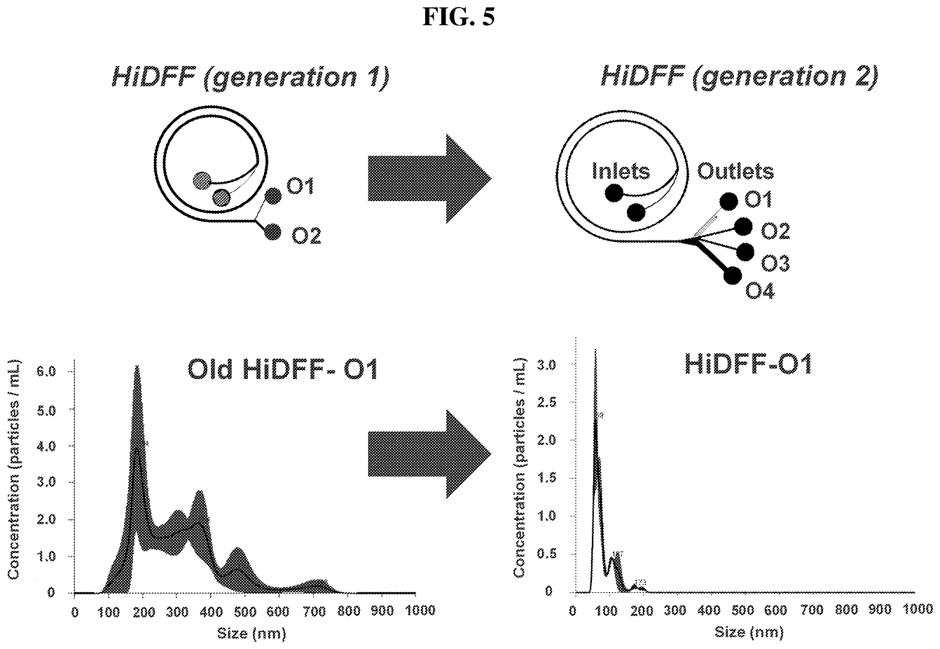

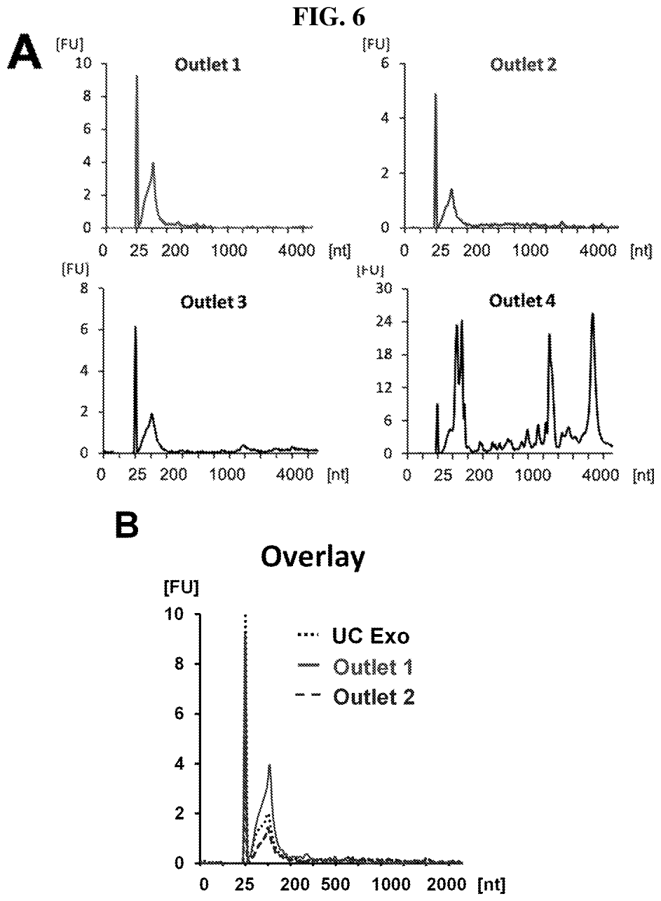

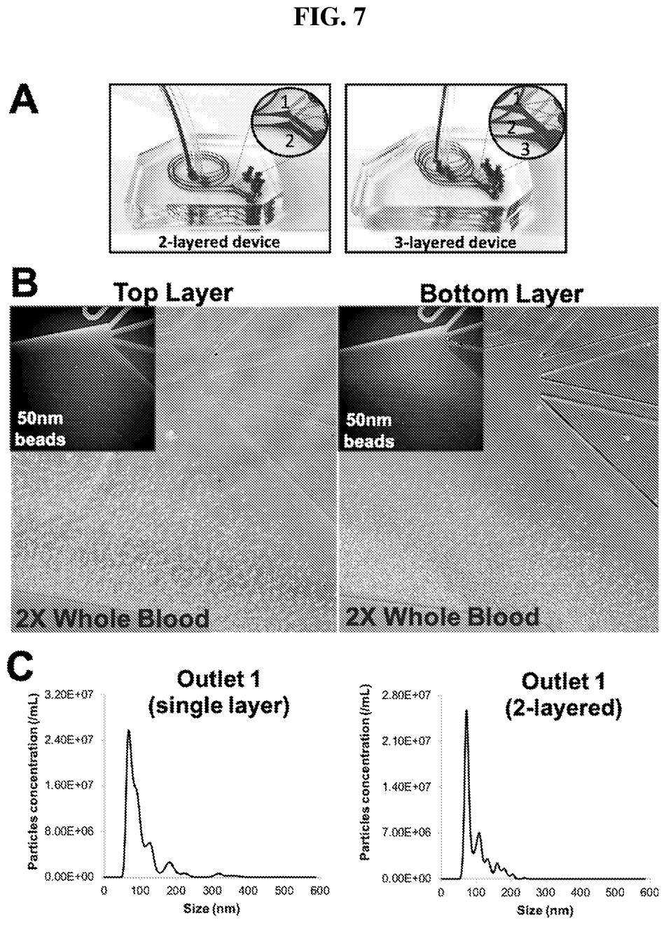

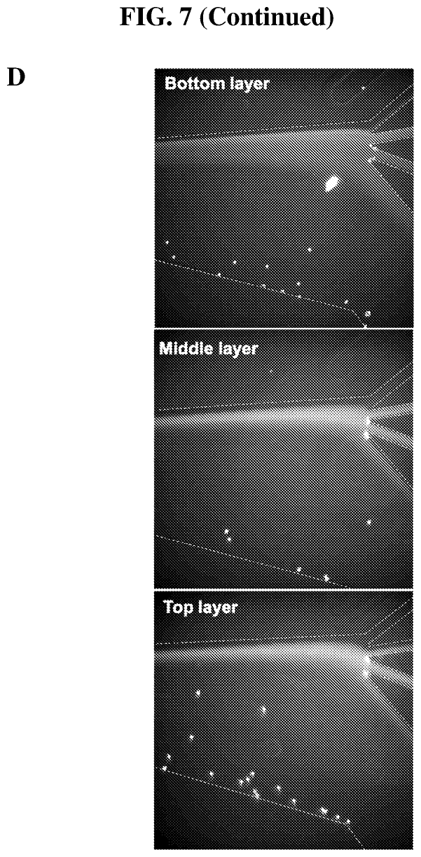

As proof-of-concept for whole blood processing, whole blood was diluted in a ratio of 1:1 with PBS and perfused into the HiDFF device. High-speed imaging clearly indicated efficient removal of platelets (˜2-3 μm) and larger blood cells (˜5-15 μm) via outlets 3 and 4, respectively (FIG. 4A). The HiDFF-sorted EVs (outlets 1, 2) were then characterized using nanoparticle tracking analysis (NTA). HiDFF outlet 1 (O1) showed a slightly lower EVs recovery than outlet 2 (O2), but had a more homogenous exosomes population (single dominant peak at ˜80 nm) and was used for further exosomal analysis (FIGS. 4B and 4C). Interestingly, ˜50% higher EVs yield was achieved in HiDFF O1 as compared to ultracentrifugation from the same starting blood volume, and western blot (exosomal marker (CD9), microvesicles/apoptotic bodies markers (annexin and histone)) also confirmed exosomes enrichment in HiDFF O1 (FIG. 4D). Compared to reported HiDFF design (i.e. a first generation device having 2-outlet without resistance channel), the present 4-outlet HiDFF design yielded a significantly smaller and more homogenous size range for outlet 1 (FIG. 5), which demonstrated the importance of the additional 2.5x resistance channel length in outlet 1 for small particle isolation. In addition, Bioanalyzer analysis indicated increased microRNA in HiDFF O1 over ultracentrifugation, which is advantageous for downstream RNA profiling (FIG. 6A). RNA denotes ribonucleic acid. To further increase processing throughput, identical HiDFF devices (2 to 3 layers) of the present disclosure were stacked to achieve ˜40 to 60 μL undiluted WB/min processing time with similar EVs separation performance (FIG. 7A).

### Example 4: Parameters of the Present Device

Non-limiting examples of certain parameters that may be used for the present device are set out below. The parameters serve to structurally describe the present device and is not intended to limit the device to such parameters.

Inlet Configuration:

Sample inlet is configured at the outer side of the spiral channel.

Sheath inlet is configured at the inner side of the spiral channel.

Sample to sheath flow rate ratio can be 1:5 to 1:50.

Channel Configuration:

Channel length: 3-10 cm

Channel width: 150-500 μm

Channel height: 30-100 μm

Channel aspect ratio (width/height):3-7

Radius of curvature: 0.3-1 cm

Operable Reynolds number range: 20-100

Operable Dean number range: 2-10

Sample can migrate along the top and bottom channel wall from the outer wall towards the inner wall region.

Outlet Configuration:

Exosome outlet can be at the inner wall of the spiral channel.

Outlet design can have more than 1 outlet and up to 10 outlets.

Exosome outlet can collect a fraction (˜0.5 to 4%) of the total volume output.

Exosome outlet width can be from 20-100 μm.

### Example 5A: Direct Isolation of Circulating Nanoscale Exosomes and Microvesicles from Whole Blood Towards Rapid Vascular Risk Profiling in Type 2 Diabetes Mellitus Using Present Device

Extracellular vesicles (EV) are mediators of intracellular communication in health and diseases. Despite significant interest in EV-based biomarkers in liquid biopsy, clinical utilities remain limited due to difficulties in isolating EV from whole blood with high yield and reproducibility. Inertial microfluidics is widely used for cell separation (˜10 to 20 μm in diameter) but remains challenging for smaller nanoparticles (<1 μm) due to negligible inertial forces for particle equilibrium focusing. The present device is a unique microfluidic separation technology for direct isolation of circulating EV from whole blood using an inertia-based method. This label-free approach enables simultaneous fractionation of nanoscale EV (exosomes, 50 to 200 nm in diameter) and medium-sized EV (microvesicles (MV), 100 nm to 1 μm in diameter) from whole blood based on differential wall-induced lift forces in spiral microchannels. Besides achieving a three-fold increase in EV yield and complete depletion of cellular constituents, the gentle sorting method also leads to a significant (ten-fold) reduction of platelet-derived MV as compared to ultracentrifugation (UC) due to minimal shear-induced platelet lysis. In a pilot clinical study of healthy (n=9) and type 2 diabetes mellitus (T2DM) (n=12) subjects, higher EV levels were detected in T2DM patients (P<0.05) using the present method, and identified a subset of “high-risk” T2DM subjects with abnormally high (˜10 to 50-fold) amount of platelet (CD41a+) or leukocyte-derived (CD45+) MV by immunophenotyping the sorted EV. In vitro endothelial cell assay further revealed that the “high-risk” T2DM EV induced significantly higher vascular inflammation (ICAM-1 expression) (P<0.05) as compared to healthy and T2DM EV, thus reflecting a pro-inflammatory phenotype. Overall, the method presented here is a scalable and versatile EV research tool which reduces manual labour, cost and processing time. This facilitates the further development of EV-based diagnostics using liquid biopsy, and a combinatory EV immuno- and functional phenotyping strategy can potentially be used for rapid vascular risk stratification in T2DM.

### Example 5B: High Throughput ExoDFF (ExoDFF^{HT})

The present method and microfluidic device are versatile to cater for another example of the multiplexing strategy disclosed herein, wherein the position of outlets and inlets were switched to create another second generation HiDFF (hereafter termed as 2^{nd} Gen ExoDFF) so that the inlets of each subunit spiral can be connected easily without modifying the outlet design, wherein the abbreviation “ExoDFF” denotes for Exosomes Dean Flow Fractionation. In other words, for the present ExoDFF device, the inlets are positioned away from the spiral channel and the outlets are positioned in a manner where the channel spirals around the outlets (e.g. surround the outlets—see center and rightmost images of FIG. 8A). Meanwhile, the present microfluidic device having a HiDFF configuration is where the inlet ports are arranged in a manner where the spiral-shaped channel horizontally spirals around the inlet ports and the at least two outlet ports are arranged away from the spiral-shaped channel. Both the present HiDFF and ExoDFF devices have the longer first outlet channel.

As proof-of-concept, 4 subunits of spiral channel were designed and fabricated to become high throughput ExoDFF (ExoDFF^{HT}) (FIG. 8A). Outlets were reduced from 4 to 2 in 2^{nd} Gen ExoDFF (left image in FIG. 8A), and from 16 to 8 in ExoDFF^{HT} (right image in FIG. 8A). Channel length of O1 in 2^{nd} Gen ExoDFF was optimized using 50 nm, 500 nm and 1 μm beads (FIG. 8B). Similar size-dependent bead separation as compared to first generation 4-outlet HiDFF was achieved with majority of 50 nm beads and few 500 nm beads eluted to O1 without contamination of 1 μm beads at optimal flow rate (Reynolds number is at 42).

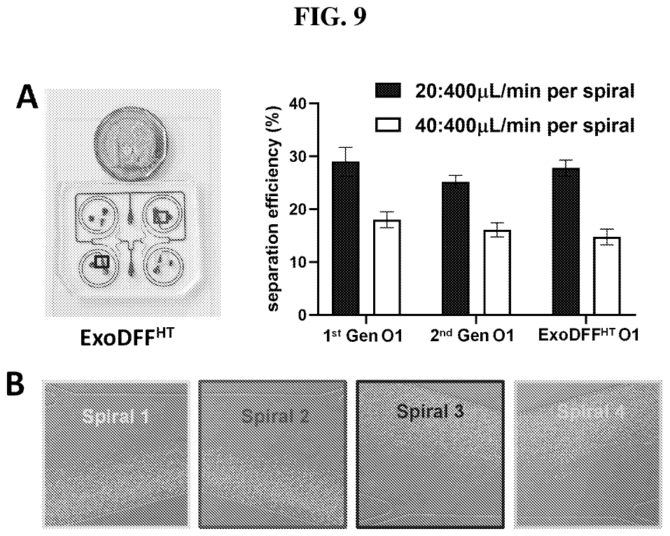

Separation performance of the present ExoDFF^{HT} was further quantified using 200 nm beads. Nanoparticle tracking analaysis (NTA) results showed that highest separation efficiency of 2^{nd} Gen ExoDFF and ExoDFF^{HT} were at 20:400 μL/min at each spiral (FIG. 9A). At sample-to-sheath flow rate of 20:400 (and 40:400) μL/min at each spiral, there was a slight reduction of separation efficiency to 25.2% (and 16.1%) and 27.8% (and 14.8%) in 2^{nd} Gen ExoDFF and ExoDFF^{HT} as compared to 1^{st} Gen HiDFF of 29% (and 18%). For whole blood processing, high speed images at each ExoDFF^{HT} subunit's outlet region also showed that larger blood cells were aligned next to outer wall and eluted into the larger waste outlet (FIG. 9B).

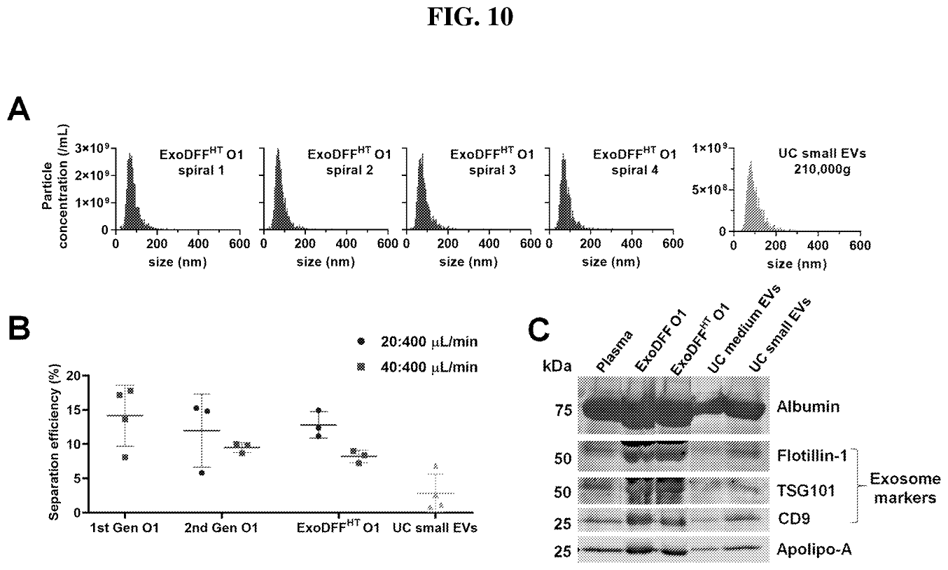

Blood borne EVs collected from 1^{st} Gen HiDFF, ExoDFF^{HT} and ultracentrifugation (UC) were next compared using NTA and Western blot. NTA showed that ExoDFF^{HT} and UC small EVs exhibited similar particle size distribution below 200 nm corresponding to small EVs (FIG. 10A). Separation efficiency ˜14% was observed in 1^{st} Gen HiDFF (40:400 μL/min), 2^{nd} Gen ExoDFF (20:400 μL/min) and ExoDFF^{HT} (20:400 μL/min) as compared to UC of ˜4%, which indicates ˜3-fold higher efficiency using various ExoDFF devices (FIG. 10B). Western blot result also showed higher expression of exosomal marker such as Flotillin-1, TSG101 and CD9 in both ExoDFF devices as compared to UC. Plasma albumin and lipoprotein (apoA-I) were not fully removed and slightly higher albumin expression in both ExoDFF devices was observed compared to UC (FIG. 10C). Taken together, ExoDFF^{HT} demonstrated 2-fold increase in throughput after multiplexing 1^{st} Gen HiDFF, which processed 40 μL whole blood per minute. Higher throughput can be achieved by through the ExoDFF^{HT} outlet length.

### Example 5C: High Throughput ExoDFF (ExoDFF^{HT}) with “Half-Loop” Design)

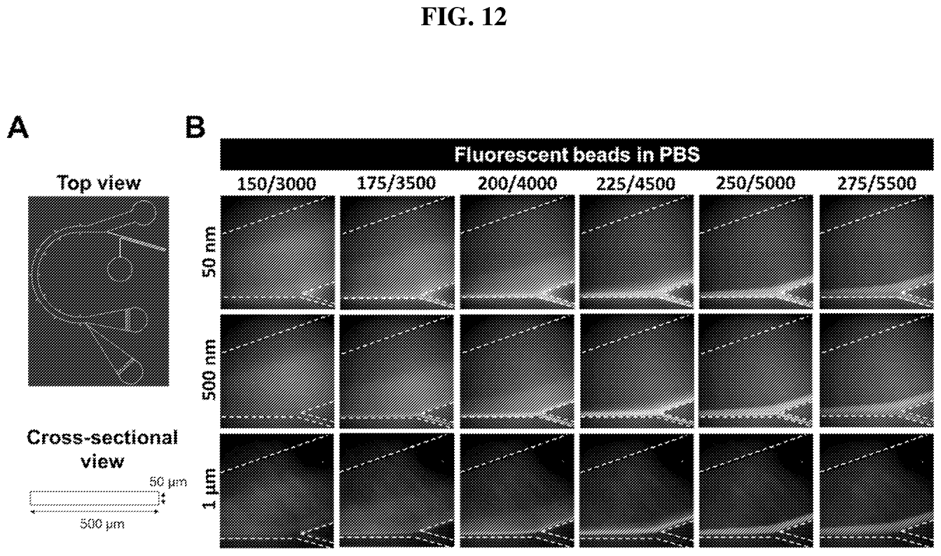

As mentioned above, the present method and microfluidic device is versatile. In another strategy of the present disclosure for high throughput sample processing, the ExoDFF spiral length was shortened by 4-5 times to a “half-loop” channel design (i.e. semi-spiral channel). The idea was to increase the flow rate to achieve similar EV sorting performance. This was demonstrated using beads (50 nm, 500 nm, 1 um) in 2 half loop designs of 300 μm width (same as ExoDFF) (FIG. 11A to 11C) and 500 μm channel width (FIGS. 12A and 12B). The data suggest that the flow rate can be increased by ˜4-5 times, and this is advantageous due to lower pressure drop in a shorter channel length. This can be applied to the present HiDFF device as well.

### Example 5D: Clinical Validation of the Present 4-Outlet Device (Type 2 Diabetes Mellitus)

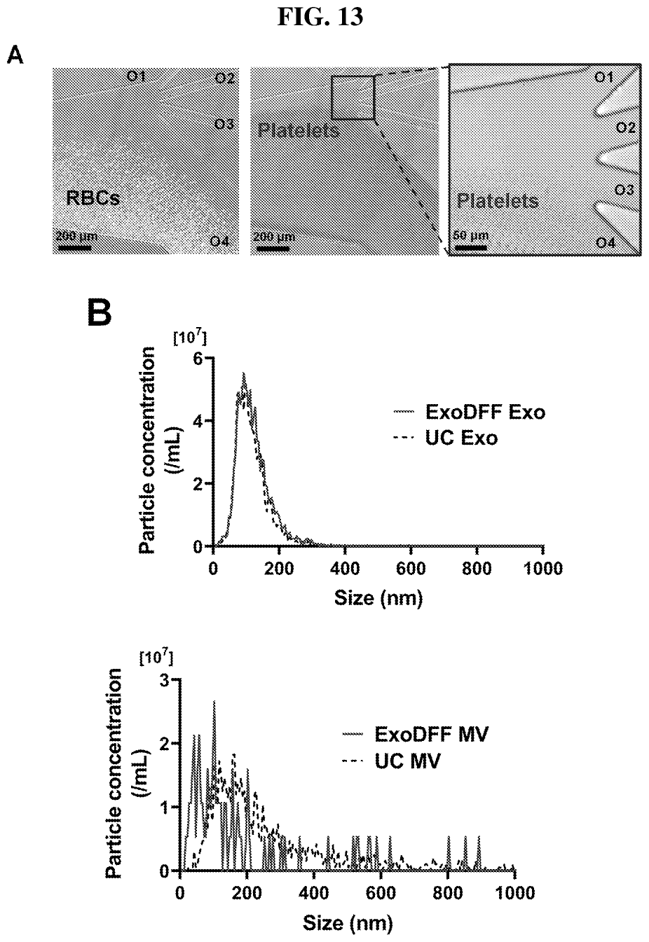

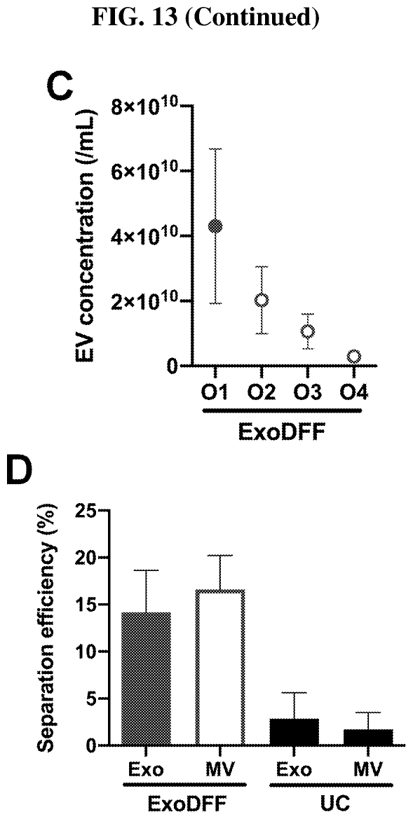

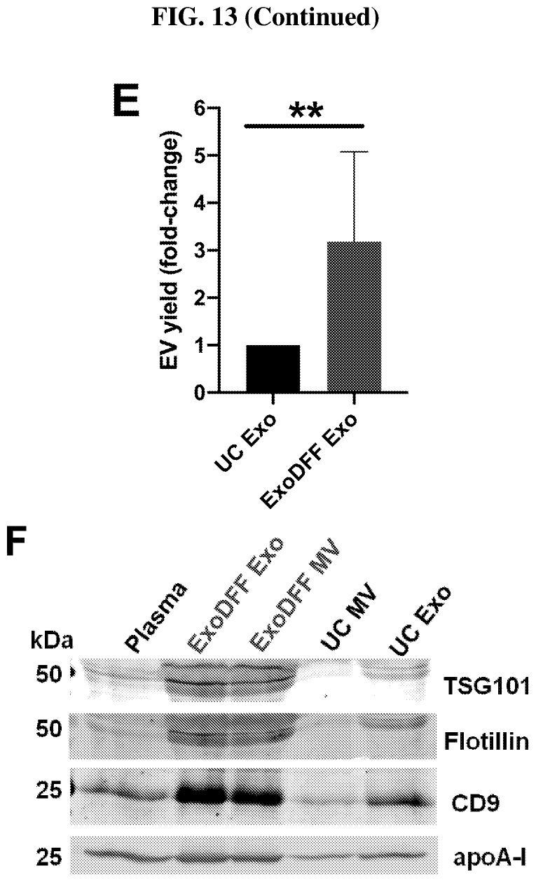

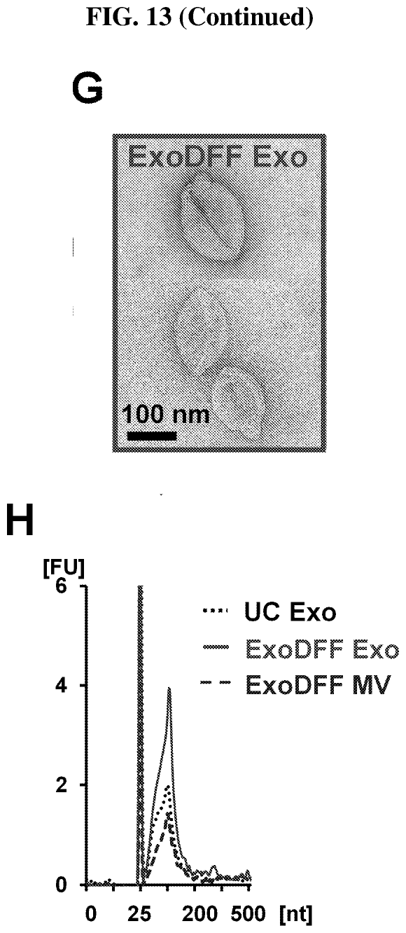

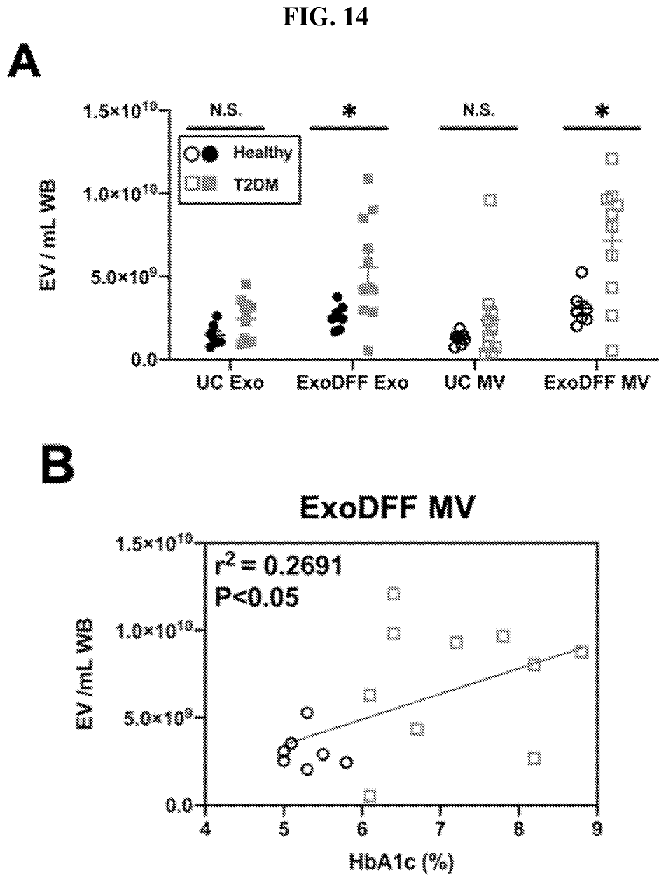

Isolation of circulating extracellular vesicles (EV) from whole blood using a 4-outlet HiDFF (hereinafter the chip may also be referred to as ExoDFF only for simplicity and to signify both the present HiDFF and ExoDFF configurations are operable for this example) was demonstrated, wherein the present 4-outlet HiDFF device has the longer first outlet channel and the inlet ports are arranged in a manner where the spiral-shaped channel spirals around the inlet ports and the outlet ports are arranged away from the spiral-shaped channel. Diluted whole blood (1:1 PBS) and sheath fluid were perfused into the ExoDFF device at the optimized flow rates (Re˜40, 40 μLmin⁻¹ (sample) and 400 μLmin⁻¹ (sheath)). Re denotes Reynolds number. As shown in high speed images, larger blood cells (˜6-15 μm) remained close to the channel outer wall prior outlets and were efficiently removed via O4 (FIG. 13A). Smaller platelets (˜2-3 μm) migrated further towards the inner wall and were sorted into both O3 and O4. Since EV are not visible using microscope imaging, the eluents from O1 and O2 were collected for downstream characterization of size distribution and particle yield using NTA. Results were also compared with multi-step ultracentrifugation (UC) which is the gold standard technique and most widely used. Both ExoDFF O1 and UC exosomes (UC exo) exhibited similar EV size distribution with a dominant peak at ˜150 nm (FIG. 13B), while ExoDFF O2 and UC MV had a wider EV size distribution (˜100-800 nm). EV concentration was the highest in ExoDFF O1 (FIG. 13C), which translates to an EV separation efficiency of ˜15 (±3.8)%, and was ˜3-fold higher as compared to UC (˜4.8 (±4.2)%) (FIG. 13D). This led to a significant improvement in EV yield in ExoDFF O1 over UC exo (P<0.05) (FIG. 13E), and clearly suggests the importance and unique ability of ExoDFF to isolate EV in a single-step manner, as opposed to UC (more prone to EV losses). To further identify exosomes from ExoDFF O1/O2 and UC samples, exosomal protein markers TSG101, Flotillin and CD9 were characterized using western blot. Consistent with NTA results, strong signals were detected in ExoDFF O1, O2 as compared to UC exo with equal sample volumes, which qualitatively suggests higher EV concentrations after ExoDFF processing (FIG. 13F). ApoA-I marker which indicates lipoprotein contamination, was faintly detected in both ExoDFF and UC samples. Transmission electron microscopy (TEM) was also performed on EV from ExoDFF O1 which revealed distinctive cup-shaped morphology of exosomes of ˜60-120 nm (FIG. 13G). Finally, the total RNA content was isolated from ExoDFF O1/O2 and UC Exo, and examined RNA size distribution and yield using Bioanalyzer RNA Pico assay. RNA from ExoDFF O1, O2 and UC exo showed short RNA profiles with a dominant peak below 200nt, which is representative of exosome-derived microRNA23 (FIG. 13H). RNA yield from ExoDFF O1 was the highest, which indicates high exosome concentration and corroborated with both NTA and western blot results. Taken together, these results clearly indicate that ExoDFF is an efficient EV isolation device which can process whole blood directly, and is superior over UC in terms of EV yield, processing time and ease of use (single-step). As a proof-of-concept for clinical testing, nanoscale EV and medium-sized EV were isolated from ExoDFF Exo and MV, respectively, in healthy (n=9) and T2DM (n=12) subjects. A comparison was also made with UC Exo and UC MV. While EV had similar size distribution regardless of the isolation methods, EV counts were higher (per unit volume of whole blood) for T2DM patients as compared to healthy subjects in ExoDFF Exo (P<0.05) and ExoDFF MV (P<0.05) (FIG. 14A). Both UC Exo and UC MV did not show any significant differences in EV count between both groups. Note that EV yields from ExoDFF Exo and MV were higher than UC Exo and UC MV, respectively, which indicates that ExoDFF is a more efficient EV separation tool in this instance. As EV in blood are shed by dysfunctional endothelial or blood cells induced by chronic hyperglycemia and low-grade inflammation in T2DM, it was sought to determine if EV count is associated with glycemic level as a measure of disease severity. Evidently, higher hemoglobin A1c (HbA1c) level (indicator of blood glucose) corresponded to higher EV count in T2DM patients in ExoDFF MV (FIG. 14B), which suggests that EV from ExoDFF MV may provide disease-specific EV signature or phenotype.

### Example 6: Commercial and Potential Applications

A low-cost and label-free microfluidic strategy is herein developed for direct and scalable isolation of circulating exosomes from whole blood. The present methods and microfluidic device are straightfoward to use, requires minimal user operation, and can be readily translated to clinical settings to accelerate exosome biology research, and development of point-of-care exosome diagnostic tools.

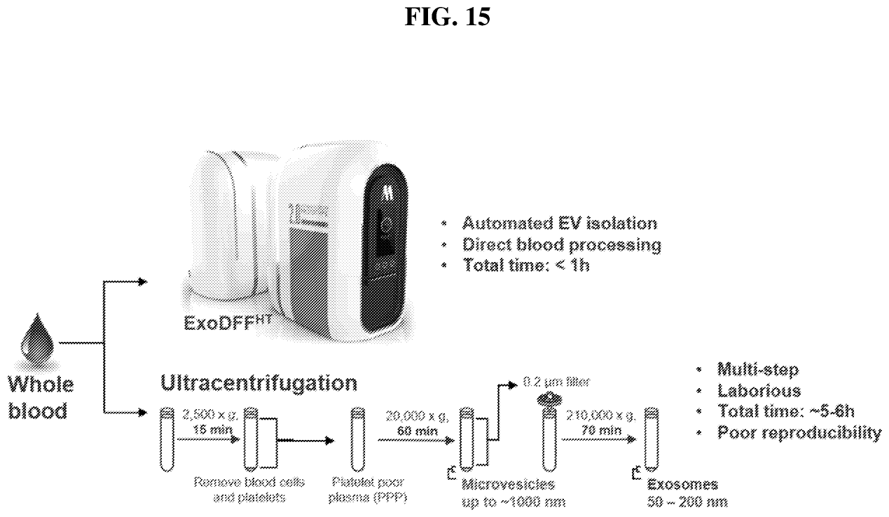

The development of a single-step, high-throughput and label-free exosome sorting method has great commercial interest ranging from genomics and proteomics studies, to large-scale exosomes biomanufacturing and point-of-care clinical diagnostics. With the present technology, it can advance the development of a “sample-in-answer-out” liquid biopsy-based testing system (FIG. 15) for rapid EVs quantitation and phenotyping and allows early detection of diseases. For exosomes engineering applications, the presently developed continuous-flow EVs purification scheme has significant advantages over the standard ultracentrifugation due to its low-cost, minimal user operation, scalability and potential to produce GMP or clinical grade EVs in a closed circuit (sterile environment) for therapeutic applications.

The advantages of the present technology include the passive separation principle, user-friendliness (only syringe pumps needed) and low-cost operation (no need for labelling or processing). Of note, the microdevice is also portable and easily integrated to other platforms for downstream detection or analysis.

While the present disclosure has been particularly shown and described with reference to specific embodiments, it should be understood by those skilled in the art that various changes in form and detail may be made therein without departing from the spirit and scope of the present disclosure as defined by the appended claims. The scope of the present disclosure is thus indicated by the appended claims and all changes which come within the meaning and range of equivalency of the claims are therefore intended to be embraced.

1. A method of isolating cellular component from a biological fluid, wherein the cellular component comprises exosomes, bacteria, platelets, or similar-sized component, the method comprising:
providing a microfluidic device comprising a spiral-shaped channel in fluid communication with (i) two inlet ports and (ii) at least two outlet ports, wherein one of the two inlet ports is proximal to an inner wall of the spiral-shaped channel and the other inlet port is proximal to an outer wall of the spiral-shaped channel, wherein at least one of the outlet ports is in fluid communication with a container configured to store isolated cellular component;
introducing a biological fluid sample into the inlet port proximal to the outer wall and introducing a sheath fluid into the inlet port proximal to the inner wall to form a diluted sample in the spiral-shaped channel;
driving the diluted sample through the spiral-shaped channel; and
recovering the cellular component in the container,
wherein the at least two outlet ports comprise a first outlet port which is in fluid communication with the container configured to store the isolated cellular component,
wherein the spiral-shaped channel in fluid communication with the first outlet port comprises a first outlet channel which connects the spiral-shaped channel to the first outlet port and is longer than other outlet channels respectively connecting the spiral-shaped channel to the other outlet ports.

2. The method of claim 1, wherein the method does not comprise a centrifugation step.

3. The method of claim 1, wherein introducing the biological fluid sample and the sheath fluid comprises introducing the sheath fluid at a higher flow rate compared to a flow rate for introducing the biological fluid sample.

4. The method of claim 1, wherein introducing the biological fluid sample and the sheath fluid comprises introducing the biological fluid sample into the inlet port proximal to the outer wall and introducing the sheath fluid into the inlet port proximal to the inner wall at a flow rate ratio of 1:5 to 1:50.

5. (canceled)

6. The method of claim 1, wherein the spiral-shaped channel is a semi-spiral-shaped channel.

7. The method of claim 1, wherein:
the two inlet ports are arranged in a manner where the spiral-shaped channel horizontally spirals around the inlet ports and the at least two outlet ports are arranged away from the spiral-shaped channel; or
the two inlet ports are arranged away from the spiral-shaped channel and the at least two outlet ports are arranged in a manner where the spiral-shaped channel horizontally spirals around the at least two outlet ports.

8. The method of claim 1, wherein driving the diluted sample comprises driving the diluted sample to flow in the spiral-shaped channel to have:
a Reynolds number ranging from 20 to 200; and
a Dean number ranging from 2 to 20.

9. The method of claim 1, wherein the first outlet channel has a length ranging from 0.5 cm to 1.5 cm.

10. (canceled)

11. The method of claim 1, wherein the spiral-shaped channel gradually expands to a width of 500 μm to 3000 μm.

12. The method of claim 1, wherein the first outlet port channel has an output flow rate of 1% to 10% of the total flow rate of all outlet channels.

13. A microfluidic device operable to isolate a cellular component from a biological fluid, wherein the cellular component comprises exosomes, bacteria, platelets, or similar-sized component, the microfluidic device comprising:
a spiral-shaped channel in fluid communication with (i) two inlet ports and (ii) at least two outlet ports, wherein one of the two inlet ports is proximal to an inner wall of the spiral-shaped channel and the other inlet port is proximal to an outer wall of the spiral-shaped channel; and
a container in fluid communication with at least one of the outlet ports, wherein the container is configured to store isolated cellular component,
wherein the at least two outlet ports comprise a first outlet port which is in fluid communication with the container configured to store the isolated cellular component,
wherein the spiral-shaped channel in fluid communication with the first outlet port comprises a first outlet channel which connects the spiral-shaped channel to the first outlet port and is longer than other outlet channels respectively connecting the spiral-shaped channel to the other outlet ports.

14. The microfluidic device of claim 13, wherein the inlet port proximal to the inner wall of the spiral-shaped channel is operable to introduce the sheath fluid at a higher flow rate than the inlet port proximal to the outer wall of the spiral-shaped channel.

15. The microfluidic device of claim 13, wherein the inlet port proximal to the outlet wall of the spiral-shaped channel and the inlet port proximal to the inner wall of the spiral-shaped channel are operable to introduce the biological fluid sample and the sheath fluid at a flow rate ratio of 1:5 to 1:50.

16. The microfluidic device of claim 13, wherein the spiral-shaped channel is defined as having:
a width ranging from 150 μm to 500 μm;
a height ranging from 30 μm to 100 μm;
a length ranging from 3 cm to 10 cm;
a width to height aspect ratio ranging from 3 to 7; or
a radius curvature ranging from 0.3 cm to 1 cm.

17. The microfluidic device of claim 13, wherein the spiral-shaped channel is a semi-spiral-shaped channel.

18. The microfluidic device of claim 13, wherein:
the two inlet ports are arranged in a manner where the spiral-shaped channel horizontally spirals around the inlet ports and the at least two outlet ports are arranged away from the spiral-shaped channel; or
the two inlet ports are arranged away from the spiral-shaped channel and the at least two outlet ports are arranged in a manner where the spiral-shaped channel horizontally spirals around the at least two outlet ports.

19. The microfluidic device of claim 13, wherein the first outlet channel has a length ranging from 0.5 cm to 1.5 cm.

20. (canceled)

21. The microfluidic device of claim 13, wherein the spiral-shaped channel gradually expands to a width of 500 μm to 3000 μm.

22. The microfluidic device of claim 13, wherein the first channel has an output flow rate of 1% to 10% of the total flow rate of all outlet channels.

23. A method of profiling diabetes mellitus, the method comprising:
providing a blood sample and introducing the blood sample into a microfluidic device comprising:
a spiral-shaped channel in fluid communication with (i) two inlet ports and (ii) at least two outlet ports, wherein one of the two inlet ports is proximal to an inner wall of the spiral-shaped channel and the other inlet port is proximal to an outer wall of the spiral-shaped channel; and
a container in fluid communication with at least one of the outlet ports, wherein the container is configured to store isolated cellular component,
wherein the at least two outlet ports comprise a first outlet port which is in fluid communication with the container configured to store the isolated cellular component, wherein the spiral-shaped channel in fluid communication with the first outlet port comprises a first outlet channel which connects the spiral-shaped channel to the first outlet port and is longer than other outlet channels respectively connecting the spiral-shaped channel to the other outlet ports;
operating the microfluidic device; and
isolating exosomes according to the method of claim 1, for profiling diabetes mellitus.
